(19) United States
(12) Patent Application Publication
(10) Pub. No.: US 2026/0315778 A1
(43) Pub. Date: Oct. 15, 2026

(54) METHODS AND SYSTEMS FOR DETECTING AN OBSTRUCTION EVENT ASSOCIATED WITH A HEART PUMP

(71) Applicant: Abiomed, Inc., Danvers, MA (US)

(72) Inventors: Mohammed Alwatban, Danvers, MA (US); Yuka Akiyama, Danvers, MA (US); Francesco Vicario, Danvers, MA (US); Samuel Brown, Danvers, MA (US); Andrew Chen, Danvers, MA (US); Omar Alamoudi, Danvers, MA (US); Michelle Graham, Danvers, MA (US); Ahmad El Katerji, Danvers, MA (US)

(73) Assignee: Abiomed, Inc., Danvers, MA (US)

(21) Appl. No.: 19/533,967

(22) Filed: Feb. 9, 2026

Related U.S. Application Data
(60) Provisional application No. 63/756,460, filed on Feb. 10, 2025.
(60) Provisional application No. 63/887,143, filed on Sep. 24, 2025.

(51) Int. Cl. A61M 60/554; A61M 60/13; A61M 60/17; A61M 60/216; A61M 60/414; A61M 60/585; G16H 40/63

(52) U.S. Cl. CPC A61M 60/554; A61M 60/13; A61M 60/17; A61M 60/216; A61M 60/414; A61M 60/585; G16H 40/63; A61M 2205/18; A61M 2205/3331; A61M 2205/502; A61M 2205/583

(57) ABSTRACT

Methods and apparatus of detecting an obstruction event associated with operation of a heart pump are provided. The method includes receiving a motor current signal associated with a motor of the heart pump, receiving a pump speed signal associated with the heart pump, detecting, using a computer processor, an obstruction event, and displaying, when an obstruction event is detected, an indication of the obstruction event on a user interface associated with the heart pump. Detecting the obstruction event is based, at least in part on a first value of the motor current signal being above a first threshold value at a first time, and a second value associated with the pump speed signal being less than a second threshold value at a second time, wherein the first time and the second time are within a time window having a length of 120 ms or less.

### CROSS REFERENCE TO RELATED APPLICATIONS

This application claims the benefit under 35 U.S.C. § 119(e) to U.S. Provisional Patent Application No. 63/756,460, filed Feb. 10, 2025, and titled, “METHODS AND SYSTEMS FOR DETECTING AN OBSTRUCTION EVENT ASSOCIATED WITH A HEART PUMP,” and U.S. Provisional Patent Application No. 63/887,143, filed Sep. 24, 2025, and titled, “METHODS AND SYSTEMS FOR DETECTING AN OBSTRUCTION EVENT ASSOCIATED WITH A HEART PUMP,” the contents of each of which is incorporated by reference herein in its entirety.

### FIELD OF THE INVENTION

This disclosure relates to techniques for detecting an obstruction event during operation of a heart pump.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span one or more heart valves. The intravascular blood pump is typically disposed at the end of the catheter. Once in position, the pump may be used to assist the heart and pump blood through the circulatory system and, therefore, temporarily reduce load on the patient's heart, such as to enable the heart to recover after a heart attack. An exemplary intravascular blood pump is available from ABIOMED, Inc., Danvers, MA under the tradename Impella® heart pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. In some instances, the controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

An intravascular blood pump designed for heart assistance can extend through the pulmonary valve and into the pulmonary artery to expel blood into the pulmonary artery. In some instances, biomaterial (e.g., a blood clot) may be ingested by the blood pump, which may reduce blood flow through the pump (e.g., by blocking the pump inlet), resulting in reduced patient support. When such an obstruction event occurs, a user (e.g., a healthcare provider) may identify that there is reduced blood flow through the pump but may be unable to readily determine whether the reduced blood flow is due to an obstruction event or some other cause (e.g., suction). As used herein the terms “obstruction” and “obstruction event” refer to ingestion of biomaterial (e.g., a blood clot) into the pump rather than, for example, a suction event during which tissue in the cardiac wall blocks the inlet of the pump. Described herein are systems and methods for detecting an obstruction event for a blood pump by analyzing signals associated with operation of the pump. The occurrence of obstruction events in blood pumps configured to provide right heart support tends to be higher than left side devices due perhaps to lower pulsatility within the right side of the heart, which may result in biomaterial being less easily passed through the pump. Described herein are techniques to detect obstruction events in a blood pump configured to provide right and/or left side heart support to a patient.

In some embodiments, a method of detecting an obstruction event associated with operation of a heart pump is provided. The method includes receiving a motor current signal associated with a motor of the heart pump, receiving a pump speed signal associated with the heart pump, detecting, using a computer processor, an obstruction event, and displaying, when an obstruction event is detected, an indication of the obstruction event on a user interface associated with the heart pump. Detecting an obstruction event is based, at least in part on a first value of the motor current signal being above a first threshold value at a first time, and a second value associated with the pump speed signal being less than a second threshold value at a second time, wherein the first time and the second time are within a first time window having a length of 120 ms or less.

In one aspect, the method further includes receiving a pressure signal from at least one pressure sensor arranged on the heart pump, and determining a first baseline shift of the pressure signal occurring within the first time window, wherein detecting an obstruction event is further based, at least in part, on a value of the first baseline shift of the pressure signal being larger than a third threshold value. In another aspect, the heart pump is positioned across a valve in a right side of a patient's heart. In another aspect, the method further includes filtering the pressure signal to obtain a filtered pressure signal, and determining the first baseline shift of the pressure signal based on the filtered pressure signal. In another aspect, filtering the pressure signal comprises filtering the pressure signal using an infinite impulse response (IIR) filter. In another aspect, the method further includes determining, based at least in part on the pressure signal, a type of obstruction event, and displaying an indication of the type of obstruction event on the user interface associated with the heart pump. In another aspect, the method further includes determining a second baseline shift of the pressure signal occurring outside of the first time window, and displaying on the user interface, an indication that the obstruction event has resolved when a value of the second baseline shift is larger than a fourth threshold value. In another aspect, the pressure signal comprises a differential pressure signal indicating a pressure across a valve in a patient's heart. In another aspect, determining a first baseline shift comprises comparing a current value of the pressure signal with a previous value of the pressure signal, and the previous value of the pressure signal occurred a predetermined number of samples prior to the current value. In another aspect, the length of the first time window is 100 ms or less. In another aspect, the length of the first time window is 80 ms or less.

In another aspect, the heart pump is positioned across a valve in a left side of a patient's heart. In another aspect, the method further includes receiving a pressure signal from a pressure sensor arranged on the heart pump, computing a first value of the pressure signal within a second time window after an end of the first time window, computing a second value of the pressure signal within a third time window before a start of the first time window, and determining a first shift of the pressure signal based, at least in part, on the first value of the pressure signal and the second value of the pressure signal, wherein detecting an obstruction event is further based, at least in part, on a value of the first shift of the pressure signal being greater than a third threshold value. In another aspect, the second time window begins an ignored time window after the end of the first time window, and the third time window ends the ignored time window before the start of first time window. In another aspect, the ignored time window is at least 30 seconds. In another aspect, the second time window and the third time window are equal lengths of time. In another aspect, computing the first value of the pressure signal within the second time window comprises determining the first value as an average minimum value of the pressure signal within the second time window, and computing the second value of the pressure signal within the third time window comprises determining the second value as an average minimum value of the pressure signal within the third time window.

In another aspect, the method further includes determining one or more derived signals based, at least in part, on the motor current signal and/or the pump speed signal, and detecting an obstruction event is further based, at least in part, on a value of the one or more derived signals. In another aspect, the one or more derived signals includes a motor current amplitude modulation signal, and the method further includes computing a first value of the motor current amplitude modulation signal within a second time window after an end of the first time window, computing a second value of the motor current amplitude modulation signal within a third time window before a start of the first time window, and determining a shift of the motor current amplitude modulation signal based, at least in part, on the first value and the second value, wherein detecting an obstruction event is further based, at least in part, on a value of the shift of the motor current amplitude modulation signal. In another aspect, the second time window begins an ignored time window after the end of the first time window, and the third time window ends the ignored time window before the start of the first time window. In another aspect, the ignored time window is at least 30 seconds. In another aspect, the second time window and the third time window are equal lengths of time. In another aspect, the one or more derived signals includes a motor current signal pulsatility signal, and detecting an obstruction event further based, at least in part, on a value of the motor current signal pulsatility signal being greater than a fifth threshold value. In another aspect, the method further includes generating a normalized motor current signal pulsatility signal based, at least in part, on the motor current signal pulsatility signal, wherein detecting an obstruction event further based, at least in part, on a value of the normalized motor current signal pulsatility signal.

In another aspect, detecting an obstruction event is further based, at least in part, on a fourth value of the motor current signal being above a sixth threshold value. In another aspect, the method further includes computing the sixth threshold value based, at least in part, on a maximum value of the motor current signal value during the first time window and a value of the motor current signal after the first time window. In another aspect, the method further includes computing the fourth value, wherein the fourth value is an average value of the motor current signal from a maximum value of the motor current signal during the first time window and a predetermined number of samples following the maximum value.

In some embodiments, a heart pump system is provided. The heart pump system includes a heart pump including a rotor, a motor configured to drive rotation of the rotor at one or more speeds, and a controller. The controller is configured to receive a motor current signal associated with the motor, receive a pump speed signal associated with the heart pump, detect an obstruction event, and display, when an obstruction event is detected, an indication of the obstruction event on a user interface associated with the heart pump. Detecting an obstruction event is based, at least in part on a first value of the motor current signal being above a first threshold value at a first time, and a second value associated with the pump speed signal being less than a second threshold value at a second time, wherein the first time and the second time are within a time window having a length of 120 ms or less.

In one aspect, the heart pump further includes at least one pressure sensor, and the controller is further configured to receive a pressure signal from at least one pressure sensor, and determine a first baseline shift of the pressure signal occurring within the time window, wherein detecting an obstruction event is further based, at least in part, on a value of the first baseline shift of the pressure signal being larger than a third threshold value. In another aspect, the heart pump is configured to be positioned across a valve in a right side of a patient's heart. In another aspect, the controller is further configured to filter the pressure signal to obtain a filtered pressure signal, and determine the first baseline shift of the pressure signal based on the filtered pressure signal. In another aspect, filtering the pressure signal comprises filtering the pressure signal using an infinite impulse response (IIR) filter. In another aspect, the controller is further configured to determine, based at least in part, on the pressure signal, a type of obstruction event, and display an indication of the type of obstruction event on the user interface associated with the heart pump. In another aspect, the controller is further configured to determine a second baseline shift of the pressure signal occurring outside of the time window, and display on the user interface, an indication that the obstruction event has resolved when a value of the second baseline shift is larger than a fourth threshold value. In another aspect, the pressure signal comprises a differential pressure signal indicating a pressure across a valve in a patient's heart. In another aspect, determining a first baseline shift comprises comparing a current value of the pressure signal with a previous value of the pressure signal, and the previous value of the pressure signal occurred a predetermined number of samples prior to the current value. In another aspect, the length of the time window is 100 ms or less. In another aspect, the length of the time window is 80 ms or less. In another aspect, the heart pump system further includes a current sensor configured to sense the motor current signal.

In some embodiments, a controller for a heart pump system is provided. The controller includes at least one hardware processor configured to receive a motor current signal associated with a motor of the heart pump system, receive a pump speed signal associated with the heart pump system, detect an obstruction event, and display, when an obstruction event is detected, an indication of the obstruction event on a user interface associated with the heart pump system. Detecting an obstruction event is based, at least in part on a first value of the motor current signal being above a first threshold value at a first time, and a second value associated with the pump speed signal being less than a second threshold value at a second time, wherein the first time and the second time are within a time window having a length of 120 ms or less.

In one aspect, the at least one hardware processor is further configured to receive a pressure signal from at least one pressure sensor of the heart pump system, and determine a first baseline shift of the pressure signal occurring within the time window, wherein detecting an obstruction event is further based, at least in part, on a value of the first baseline shift of the pressure signal being larger than a third threshold value. In another aspect, the heart pump system includes a heart pump configured to be positioned across a valve in a right side of a patient's heart. In another aspect, the at least one hardware processor is further configured to filter the pressure signal to obtain a filtered pressure signal, and determine the first baseline shift of the pressure signal based on the filtered pressure signal. In another aspect, filtering the pressure signal comprises filtering the pressure signal using an infinite impulse response (IIR) filter. In another aspect, the at least one hardware processor is further configured to determine, based at least in part, on the pressure signal, a type of obstruction event, and display an indication of the type of obstruction event on the user interface associated with the heart pump system. In another aspect, the at least one hardware processor is further configured to determine a second baseline shift of the pressure signal occurring outside of the time window, and display on the user interface, an indication that the obstruction event has resolved when a value of the second baseline shift is larger than a fourth threshold value. In another aspect, the pressure signal comprises a differential pressure signal indicating a pressure across a valve in a patient's heart. In another aspect, determining a first baseline shift comprises comparing a current value of the pressure signal with a previous value of the pressure signal, wherein the previous value of the pressure signal occurred a predetermined number of samples prior to the current value. In another aspect, the length of the time window is 100 ms or less. In another aspect, the length of the time window is 80 ms or less.

In some embodiments, a method of detecting an obstruction event associated with operation of a heart pump is provided. The method includes receiving a motor current signal associated with a motor of the heart pump, receiving a pump speed signal associated with the heart pump, receiving a pressure signal from a pressure sensor arranged on the heart pump, determining one or more derived signals based, at least in part, on the motor current signal, the pump speed signal and/or the pressure signal, wherein the one or more derived signals includes a motor current amplitude modulation signal, and a motor current signal pulsatility signal, computing a first value within a first time window of the pressure signal, computing a second value within a second time window of the pressure signal, computing a third value within the first time window of the motor current amplitude modulation signal, computing a fourth value within the second time window of the motor current amplitude modulation signal, determining a first shift of the pressure signal based, at least in part, on the first value of the pressure signal and the second value of the pressure signal, determining a second shift of the motor current amplitude modulation based, at least in part, on the third value of the motor current amplitude modulation and the fourth value of the motor current amplitude modulation signal, generating a normalized motor current signal pulsatility signal based, at least in part, on the motor current signal pulsatility signal, detecting, using a computer processor, an obstruction event based, at least in part on a first value of the motor current signal being above a first threshold value at a first time, a second value associated with the pump speed signal being less than a second threshold value at a second time, wherein the first time and the second time are within a third time window having a length of 120 ms or less, a third value associated with the normalized motor current signal pulsatility signal being greater than a third threshold value, a fourth value of the motor current signal being above a fourth threshold value, wherein the fourth value is a value of the motor current signal during the third time window, a value of the first shift of the pressure signal being greater than a fifth threshold value, and a value of the second shift of the motor current amplitude modulation signal being greater than a sixth threshold value. The method further includes displaying, when an obstruction event is detected, an indication of the obstruction event on a user interface associated with the heart pump.

### BRIEF DESCRIPTION OF DRAWINGS

FIG. 1A shows an illustrative cardiac support device that may be used with some embodiments.

FIG. 1B shows an illustrative cardiac support system that includes the cardiac support device of FIG. 1A.

FIG. 2A shows a first set of plots of various pump signals that may be used to detect an obstruction event, in accordance with some embodiments.

FIG. 2B shows a second set of plots of various pump signals that may be used to detect an obstruction event, in accordance with some embodiments.

FIG. 3A is a first flowchart of a process for detecting an obstruction event associated with a heart pump, in accordance with some embodiments.

FIG. 3B is a second flowchart of a process for detecting an obstruction event associated with a heart pump, in accordance with some embodiments.

FIG. 4 illustrates an example of a user interface for providing an indication of a detected obstruction event, in accordance with some embodiments.

### DETAILED DESCRIPTION

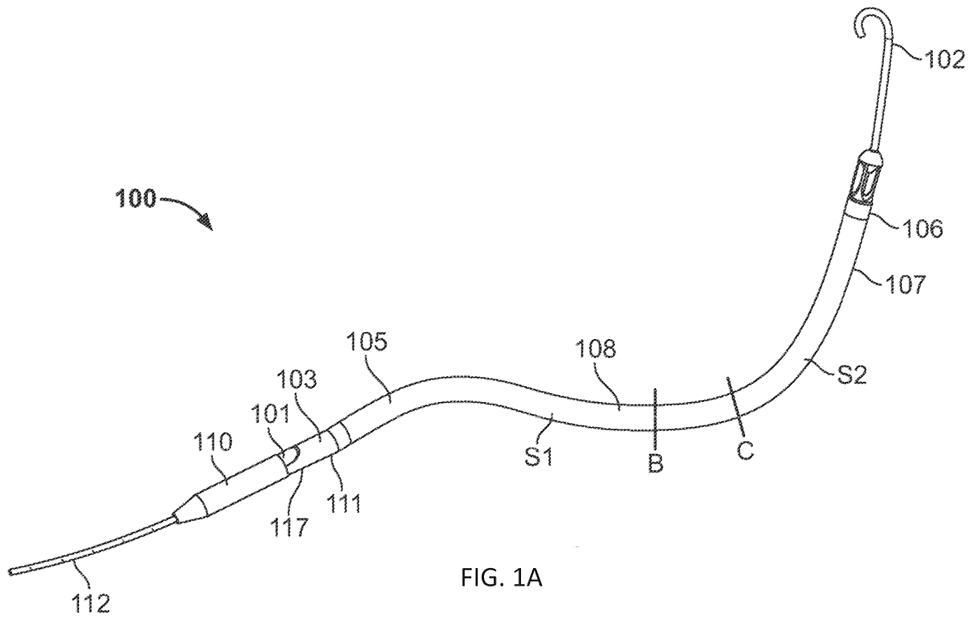

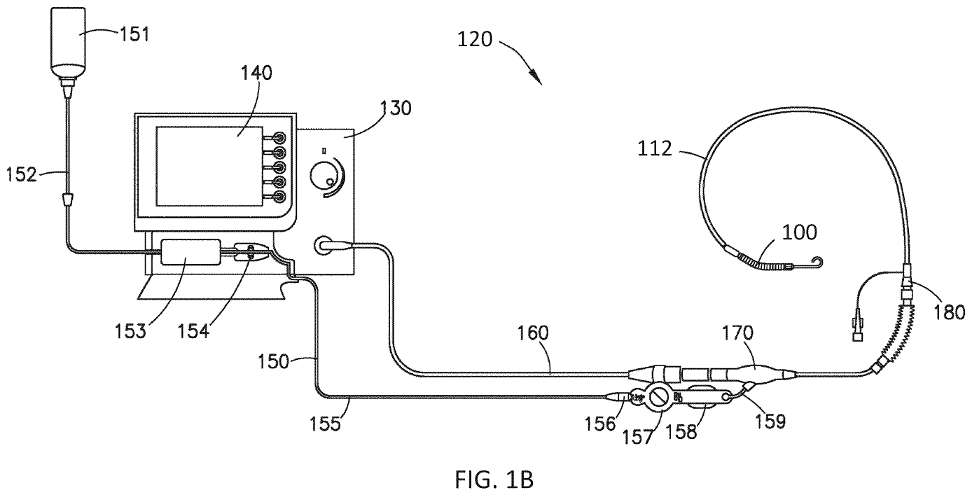

A cardiac support device (e.g., an intravascular blood pump) when properly positioned in a patient's heart may be controlled to pump blood from an inlet of the pump located on a first side of a valve in the patient's heart to an outlet of the pump located on a second side of the valve. In this way, the pump when in operation may at least partially replace or supplement the pumping action of the patient's native heart function. Occasionally blood clots or other biomaterial may form in a patient's circulatory system and in some instances such biomaterial may be ingested into the inlet of the cardiac support device, which may obstruct proper pump operation. When this happens, the patient may not receive adequate support from the cardiac support device due to reduced blood flow through the pump. Additionally, blood cells (e.g., red blood cells) in the ingested biomaterial may undergo hemolysis as they are passed through the pump, which may have negative health implications for the patient. The presence of an obstruction in the pump may manifest to a user as reduced blood flow though the pump displayed, for example on display 140 of controller 130 associated with cardiac support system 120 shown in FIG. 1B. When a user identifies that there is reduced blood flow through the heart pump, the user may manually troubleshoot the cause of the reduced flow to determine if it is due to an obstruction event (e.g., ingestion of biomaterial into the pump) or due to some other factor (suction). Determining the cause of the reduced blood flow may be important to enable a healthcare provider to take appropriate action to resolve the cause of the reduced blood flow, thereby ensuring that the patient is provided with sufficient cardiac support. The inventors have recognized and appreciated that one or more signals associated with the operation of the cardiac support device may be analyzed to automatically detect obstruction events, thereby reducing the need for users to perform manual troubleshooting when reduced flow through the device is identified.

FIG. 1A shows an illustrative embodiment of a blood pump assembly 100 according to the present disclosure. The blood pump assembly 100 may include a pump 101, a pump housing 103, a proximal end 105, a distal end 107, a cannula 108, an impeller (not shown), an atraumatic extension 102, a catheter 112, an inlet area 110, an outlet area 106, and blood exhaust apertures 117. The catheter 112 may be connected to the inlet area 110 of the cannula 108 in some embodiments. The inlet area 110 may be located near the proximal end 105 of the cannula, and the outlet area 106 may be located toward the distal end 107 of the cannula 108. The inlet area 110 may include a pump housing 103 with a peripheral wall 111 extending about a rotation axis of the impeller blades, positioned radially outward of the inner surface with respect to the rotation axis of the impeller. The impeller may be rotatably coupled to the pump 101 at the inlet area 110 adjacent to the blood exhaust apertures 117 formed in the peripheral wall 111 of the pump housing 103. The pump housing 103 may be composed of a metal in accordance with some implementations. The atraumatic extension 102, also referred to as a “pigtail,” may be connected to the distal end 107 of the cannula 108 and may assist with stabilizing and/or positioning the blood pump assembly 100 into the correct position in the heart. The atraumatic extension 102 may be configurable from a straight to a partially curved configuration. The atraumatic extension 102 may be composed, at least in part of a flexible material, and may have dual stiffness. It should be appreciated that some embodiments of the pump assembly may not include atraumatic extension 102.

The cannula 108 may have a shape which matches (or is similar to) the anatomy of the right ventricle of a patient. In the exemplary embodiment shown in FIG. 1A, the cannula has a proximal end 105 arranged to be located near the patient's inferior vena cava, and a distal end 107 arranged to be located near the pulmonary artery. The cannula 108 may include a first segment Sl extending from the inflow area to a point B between the inlet area 110 and the outlet area 106. The cannula 108 may also include a second segment S2 extending from a point C, which is between the inlet area 110 and the outlet area 106, to the outlet area 106. In some implementations, points B and C may be located at the same location along cannula 108. The first segment Sl of the cannula may form an ‘S’ shape in a first plane. In some implementations, segment Sl can have curvatures between 30 degrees and 180 degrees. The second segment S2 of the cannula may form an ‘S’ shape in a second plane. In some implementations, segment S2 can have curvatures between 30 degrees and 180 degrees (e.g., 40°, 50°, 60°, 70°, 80°, 90°, 100°, 110°, 120°, 130°, 140°, 150°, 160°, or) 170°. The second plane can be different from the first plane. In some implementations, the second plane may be parallel or identical to the first plane.

Although shown with an ‘S’ shape, it will be appreciated that other implementations of the blood pump assembly may be formed with other shapes (e.g., a ‘U’ shape), or with no shape at all when outside the body. In such implementations, the cannula may be formed of a flexible material such that the cannula may bend during insertion and achieve the desired shape once inside the heart of the patient.

In some implementations, the blood pump assembly 100 may be inserted percutaneously through the internal jugular vein, though the right atrium and into the right ventricle. When properly positioned, the blood pump assembly 100 may deliver blood from the inlet area 110, which sits inside the patient's right atrium, through the cannula 108, to the blood exhaust apertures 117 of the pump housing 103 positioned in the pulmonary artery. Alternatively, in some implementations the blood pump assembly 100 may be inserted percutaneously through the femoral artery and into the left ventricle to deliver blood from the left ventricle into the aorta.

FIG. 1B shows that blood pump assembly 100 may form part of a cardiac support system 120. Cardiac support system 120 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of blood pump assembly 100. During operation, purge subsystem 150 may be configured to deliver a purge fluid to blood pump assembly 100 through catheter 112 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between blood pump assembly 100 and controller 130. Plug 170 may connect catheter 112, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition blood pump assembly 100 in the patient's heart (e.g., by holding a position of the pump assembly relative to the patient).

As shown in FIG. 1B, in some embodiments, the cardiac support system 120 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 120 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to blood pump assembly 100. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 may include a flexible rubber diaphragm that provides the additional filling volume by means of an expansion chamber. Infusion filter 158 may help prevent bacterial contamination and air from entering catheter 112. Sidearm 159 may provide a fluidic connection between infusion filter 158 and plug 170. Although shown as having separate purge tubing and connector cable, it will be appreciated that in some embodiments, the cardiac support system 120 may include a single connector with both fluidic and electric lines connectable to the controller 130.

During operation, controller 130 may be configured to control the electrical power delivered to the motor of the blood pump assembly 100 (e.g., via connector cable 160), thereby controlling the speed of the motor. Different motor speeds may be referred to herein as different “P-levels” with a P-level of P-1 corresponding to a minimum speed of the motor and a P-level of P-9 corresponding to a maximum speed of the motor. Blood pump assembly 100 may include a current sensor (not shown) configured sense motor current associated with an operating state of the motor, and controller 130 may be configured to receive the output of the current sensor as a motor current signal. Controller 130 may be configured to detect an obstruction event based, at least in part, on the motor current signal and a pump speed signal based on a measured speed of the pump, as described in more detail herein. The current sensor may be included in controller 130 or may be located along any portion of the connector cable 160 between controller 130 and the motor. Additionally or alternatively, the current sensor may be included in the motor and controller 130 may be configured to receive the motor current signal via a data line coupled to controller 130 and the motor.

In some embodiments, controller 130 may be configured to receive measurements from one or more pressure sensors (not shown) included as a portion of blood pump assembly 100 and purge disc 154. Controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter 112 and plug 170. Sensor cables (not shown) within catheter 112, connector cable 160, and plug 170 may provide an electrical connection between components of the blood pump assembly 100 (e.g., one or more pressure sensors) and controller 130. Motor cables (not shown) within catheter 112, connector cable 160, and plug 170 may provide an electrical connection between the motor of the blood pump assembly 100 and controller 130. During operation, controller 130 may be configured to receive measurements from one or more pressure sensors of the blood pump assembly 100 through the sensor cables (e.g., optical fibers) and to control the electrical power delivered to the motor of the blood pump assembly 100 through the motor cables. By controlling the power delivered to the motor of the blood pump assembly 100, controller 130 may be operable to control the speed of the motor.

Various modifications can be made to cardiac support system 120 and one or more of its components. For instance, one or more additional sensors may be added to blood pump assembly 100. In another example, a signal generator may be added to blood pump assembly 100 to generate a signal indicative of the rotational speed of the motor of the blood pump assembly 100. As another example, one or more components of cardiac support system 120 may be separated. For instance, display 140 may be incorporated into another device in communication with controller 130 (e.g., wirelessly or through one or more electrical cables).

As described herein, a cardiac support system (e.g., cardiac support system 120 shown in FIG. 1B) may include one or more sensors configured to sense data associated with operation of a heart pump. For instance, a current sensor may be configured to sense a motor current signal associated with operation of the motor of the heart pump, a speed sensor may be configured to sense a pump speed associated with operation of the rotor of the heart pump, and one or more pressure sensors may be configured to detect a pressure near an inlet of the heart pump where blood is ingested and/or an outlet of the heart pump where blood is expelled. For instance, when a right heart cardiac support device is positioned properly, the outlet of the heart pump may be positioned within the pulmonary artery of a patient's heart, and a pressure sensor arranged near the outlet of the heart pump may measure the pressure within the pulmonary artery. The pressure signal may be used, at least in part, to determine a blood flow rate through the heart pump when in operation. For instance, the pressure signal may be used in combination with a motor current signal received from a current sensor and a set of stored values to determine a flow rate of blood through the heart pump. For a right heart cardiac support device, the differential pressure between the right atrium and the pulmonary artery may also indirectly be determined based on the pressure signal measuring the pressure in the pulmonary artery and the set of stored values. Alternatively, a differential pressure between the right atrium and the pulmonary artery may be determined using a differential pressure sensor or multiple pressure sensors including one located at an inflow region of the heart pump and another located at an outflow region of the heart pump.

An obstruction event in which a biomaterial (e.g., a blood clot) is ingested into the inflow region of a heart pump during operation may be transient and consequently hard to detect when observing sensor signal plots and/or values displayed on a display (e.g., display 140) of a controller (e.g., controller 130). For example, a “signature” of an obstruction event may include presence of a fast (e.g., on the order of ms) spike in motor current that occurs simultaneous (or near simultaneous) with a change in differential pressure (e.g., as may be reflected in the pulmonary artery pressure (PAP) signal for a right sided device). In some embodiments, a “signature” of an obstruction event may include presence of a fast (e.g., on the order of ms) spike in motor current in combination with one or more of a decrease in motor speed, a spike in motor current pulsatility, or a shift in a motor current modulation signal and/or a pressure signal. In other embodiments, a “signature” of an obstruction event may include a decrease in motor current amplitude. In some embodiments, an analysis (e.g., a time-based analysis) of multiple sensor signals associated with pump operation may be used to automatically detect an obstruction event during operation of a heart pump, and an indication of the detected obstruction event may be presented to a user to enable the user to take an appropriate action to address the obstruction event. As will be appreciated, any combination of signatures mentioned herein may be used to indicate an obstruction event.

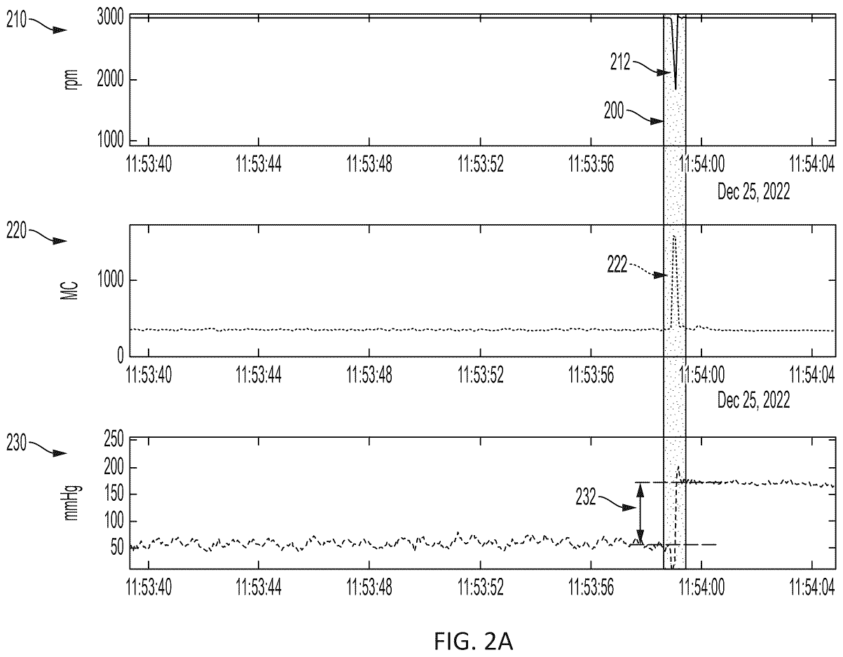

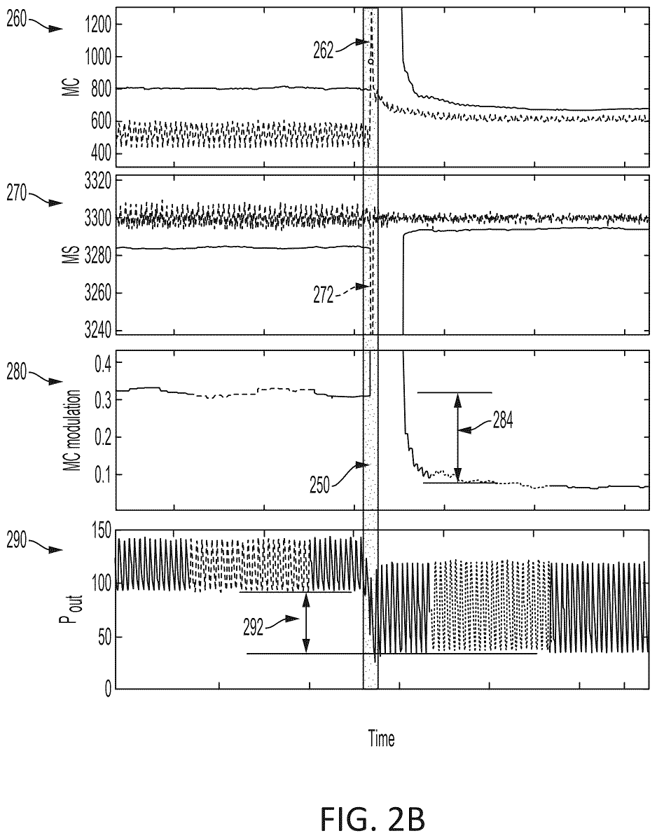

FIG. 2A illustrates example plots for a pump speed signal 210, a motor current signal 220, and a pressure signal 230 that may be sensed during operation of a heart pump, in accordance with some embodiments. Time window 200 highlights a time period during which an obstruction event may be detected, in accordance with some embodiments. As shown in FIG. 2A, the pump speed signal 210 shows a spike 212 (a sharp decrease in the pump speed signal 210) within time window 200. Additionally, also observed within time window 200, the motor current signal 220 shows a spike 222 (a sharp increase in the motor current signal 220), and the pressure signal 230 exhibits a DC shift (also referred to herein as a “baseline shift”) 232. As can be appreciated from the example shown in FIG. 2A, the events within time window 200 may occur over a relatively short timescale. For example, time window 200 may be less than or equal to 150 ms, less than or equal to 100 ms, less than or equal to 80 ms, etc. By selecting a narrow time window for analysis, and thereby requiring some or all of the aforementioned events to occur within the narrow time window to detect an obstruction event, the number of false positive detections of obstruction events may be reduced.

FIG. 2B illustrates example plots for a motor current signal 260, a pump speed signal 270, and a pressure signal 290 that may be sensed during operation of a heart pump, in accordance with some embodiments. FIG. 2B also illustrates an example plot for a motor current modulation signal 280, which may be derived from the motor current signal 260. Time window 250 highlights a time period during which an obstruction event may be detected, in accordance with some embodiments. As shown in FIG. 2B, within time window 250, the motor current signal 260 shows a spike 262 (a sharp increase in motor current signal 260) and the pump speed signal 270 shows a spike 272 (a sharp decrease in the pump speed signal 270). Additionally, a shift 284 in the value (e.g., average value, average minimum value, average maximum value) of the motor current modulation signal 280 and a shift 292 in the value (e.g., average value, average minimum value, average maximum value) of the pressure signal 290 when comparing time periods before and after time window 250 is observed. As can be appreciated from the example shown in FIG. 2B, the events within time window 250 may occur over a relatively short timescale as described herein. For example, time window 250 may be less than or equal to 150 ms, less than or equal to 100 ms, less than or equal to 80 ms, etc. By selecting a narrow time window for analysis and thereby requiring some of the aforementioned events to occur within the narrow time window to detect an obstruction event, the number of false positive detections of obstruction events may be reduced.

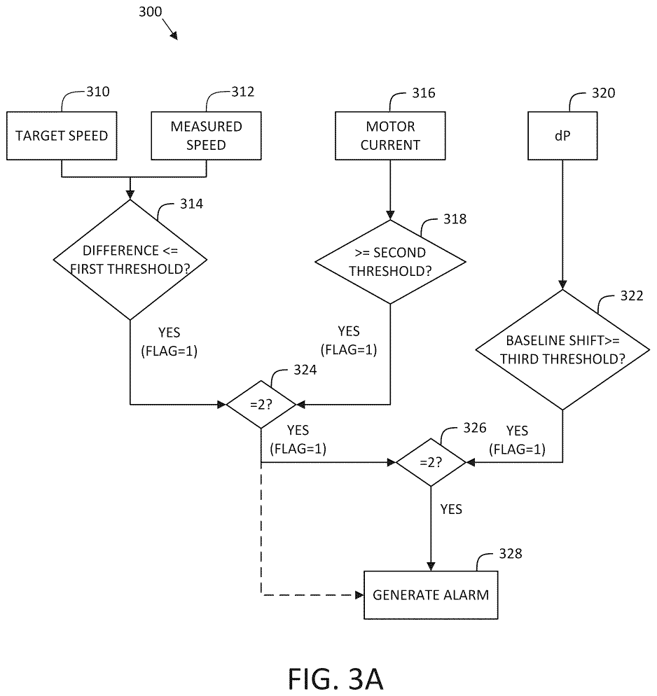

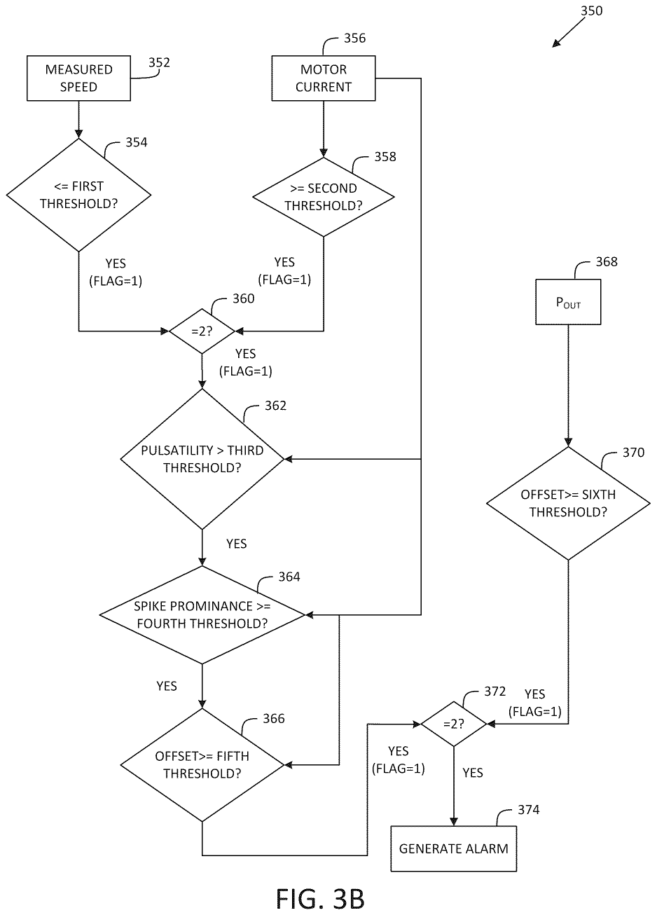

FIG. 3A illustrates a process 300 for detecting an obstruction event associated with operation of a heart pump, in accordance with some embodiments. In process 300, a pump speed signal may be determined based on a target pump speed 310 and a measured pump speed 312. For instance, the cardiac support device may include a speed sensor configured to measure a current speed of the motor. Alternatively, the cardiac support device may include one or more other sensors from which motor/pump speed may be determined. As should be appreciated, the terms “pump speed” and “motor speed” may be used interchangeably herein. As shown in act 314, when the difference between the measured pump speed 312 and the target pump speed 310 (i.e., measured speed-target speed) is less than (or less than or equal to) a first threshold value, a first flag may be set. The first flag may represent that a decrease in pump speed has exceeded the first threshold value, an example of which is shown as spike 212 in pump speed signal 210 shown in FIG. 2A. The first threshold value may be determined in any suitable way. For example, the first threshold value may be empirically determined to minimize a number of false positives. In some embodiments, the first threshold value may represent a 20% decrease in speed, a 15% decrease in speed, a 10% decrease in speed, or some other threshold value. In some embodiments, the first threshold value used in act 314 may depend, at least in part, on the motor speed of the heart pump. For instance, a different (e.g., larger) first threshold value may be used for faster pump speeds (e.g., P7-P9) compared with slower pump speeds (e.g., P5-P6).

Continuing with process 300, a motor current signal 316 may be received from a current sensor associated with a motor of the heart pump. As shown in act 318, when the value of the motor current signal 316 is greater than (or greater than or equal to) a second threshold value, a second flag may be set. The second flag may represent that an increase in motor current has exceeded the second threshold value, an example of which is shown as spike 222 in motor current signal 220 shown in FIG. 2A. The second threshold value may be determined in any suitable way. For example, the second threshold value may be empirically determined to minimize a number of false positives. In some embodiments, the second threshold value may be set at a value of 800 mA, 1000 mA, 1200 mA, 1400 mA, or some other suitable value. In some embodiments, the second threshold value used in act 318 may depend, at least in part, on the motor speed of the heart pump.

In some embodiments, when both the first flag and the second flag are set within a particular time window (e.g., 100 ms), it may be determined in act 324 that an obstruction event has been detected, and process 300 may proceed to act 328, where an indication of the obstruction event may be displayed on a user interface associated with the heart pump (e.g., an alarm may be generated and displayed on the user interface). Alternatively, as shown in process 300, in some embodiments, detection of an obstruction event may also depend on observing a baseline shift in a pressure signal (e.g., a differential pressure signal) within the same time window in which the motor current signal spike and pump speed signal spike are observed.

As shown in process 300, a pressure signal (e.g., dP) 320 may be received from at least one pressure sensor arranged on the heart pump. As shown in act 322, when a baseline shift of the pressure signal 320 is greater than (or greater than or equal to) a third threshold value, a third flag may be set. The baseline shift used in act 322 may be determined in any suitable way. For instance, in some embodiments, a comparison of a current pressure signal value at time n and a previous pressure signal value at time n−N (e.g., dP(n)−dP(n−500), where N is 500 samples) may be used to determine the baseline shift. In some embodiments, the length of the time window (i.e., N) over which the baseline shift is determined may depend, at least in part, on the sampling rate of the pressure signal. In some embodiments, the baseline shift may be determined only after a certain amount of time has passed (e.g., 20 seconds) after initial activation of the pump and/or after the pump speed has been changed (e.g., from one P-level to another P-level) to enable the pressure signal to settle to a stable baseline value.

In some embodiments, the pressure signal 320 may be filtered prior to determining the baseline shift in act 322. In some embodiments, the pressure signal 320 may be filtered using an infinite impulse response (IIR) filter to reduce the pulsatility in the pressure signal 320. The filtered pressure signal may then be used to determine the baseline shift used in act 322. The third flag may represent that the baseline shift has exceeded the third threshold value, an example of which is shown as baseline shift 232 in pressure signal 230 shown in FIG. 2A. The third threshold value may be determined in any suitable way. For example, the third threshold value may be empirically determined to minimize a number of false positives. In some embodiments, the third threshold value may represent a baseline shift of 10 mmHg, 15 mmHg, 20 mmHg, or any other suitable value. In some embodiments, the third threshold value used in act 322 may depend, at least in part, on the motor speed of the heart pump.

As shown in process 300, when the first, second and third flags are all set within a particular time window (e.g., 100 ms), it may be determined in act 326 that an obstruction event has been detected, and process 300 may proceed to act 328, where an indication of the obstruction event may be displayed on a user interface associated with the heart pump (e.g., an alarm may be generated and displayed on the user interface). Selecting a narrow time window (e.g., 120 ms, 100 ms, 80 ms, etc.) within which acts 314, 318 and 322 are performed may reduce false positives when detecting obstruction events using the techniques described herein.

In some embodiments, one or more characteristics of the pressure signal may be used to determine a type of obstruction event that has occurred. For example, if the obstruction event was a passthrough event in which the biomaterial passed through the heart pump, the baseline shift may be transient. Alternatively, if the obstruction event caused a continuous blockage of the pump operation, the baseline shift may be sustained. In some embodiments, after a first baseline shift in the pressure signal is used to detect an obstruction event, a second baseline shift in the pressure signal may be used to determine that the obstruction has been dislodged from the pump. Determining the type of obstruction event may be helpful in providing guidance to a user. For instance, when it is determined that a passthrough obstruction event has occurred, an indication of the obstruction event may be provided to the user, and the pump may continue to be used. By contrast, when it is determined that the obstruction event has caused a continuous blockage of the pump operation, an indication may be provided to the user that the patient may not be receiving sufficient support and the pump should be removed/replaced.

FIG. 3B illustrates a process 350 for detecting an obstruction event associated with operation of a heart pump, in accordance with some embodiments. Process 350 may begin in act 352 in which a measured pump speed is determined. For instance, the measured pump speed may be sensed by a speed sensor configured to directly determine the speed of the motor or one or more sensors configured to sense quantities associated with pump operations that may be used to indirectly determine the motor/pump speed. Process 350 may then proceed to act 354, in which it is determined whether the measured pump speed is less than (or less than or equal to) a first threshold value. If it is determined in act 354 that the measured pump speed is less than (or less than or equal to) the first threshold value, a first flag may be set. The first flag may represent that a decrease in pump speed (e.g., within time window 250 shown in FIG. 2B, also referred to herein as the “analysis time window”) has exceeded the first threshold value, an example of which is shown as spike 272 in pump speed signal 270 shown in FIG. 2B. The first threshold value may be determined in any suitable way. For example, in some embodiments, the first threshold value may be determined using the following equation:

Thr(w)=μ(MS(w))−Thr_{Factor}×σ(MS(w))

where, Thr(w) is the first threshold value determined over a window w having a fixed length (e.g., 5 seconds), μ(MS(w)) is a mean value of the motor speed for the window w, σ(MS(w)) is a standard deviation of the motor speed for the window w and Thr_{Factor} is a threshold factor. The threshold factor may be any suitable positive value, for example, 4, 5, 6, 7, etc. In some embodiments, Thr(w) may be calculated for a sliding overlapping windows w continuously determined during operation of the pump. In some embodiments, the first threshold value may represent a 20% decrease in speed, a 15% decrease in speed, a 10% decrease in speed, or some other threshold value. In some embodiments, the first threshold value used in act 354 may depend, at least in part, on the motor speed of the heart pump determined in act 352. For instance, a different (e.g., larger) first threshold value may be used for faster pump speeds than the first threshold value used for slower pump speeds.

Continuing with process 350, in act 356, a motor current signal may be received from a current sensor associated with a motor of the heart pump. Process 350 may then proceed to act 358, where it may be determined whether the value of the motor current signal is greater than (or greater than or equal to) a second threshold value. If it is determined in act 358 that the motor current signal is greater than (or greater than or equal to) the second threshold value, a second flag may be set. The second flag may represent that an increase in motor current has exceeded the second threshold value, an example of which is shown as spike 262 in motor current signal 260 shown in FIG. 2B. The second threshold value may be determined in any suitable way. For instance, the second threshold value may be determined using the following equation:

Thr(w)=μ(MC(w))+Thr_{Factor}×σ(MC(w))

where, Thr(w) is the second threshold value determined over a window w having a fixed length (e.g., 5 seconds), μ(MC(w)) is a mean value of the motor current for the window w, σ(MC(w)) is a standard deviation of the motor current for the window w and Thr_{Factor} is a threshold factor. The threshold factor may be any suitable positive value, for example, 4, 5, 6, 7, etc. In some embodiments, the second threshold value used in act 358 may depend, at least in part, on the motor speed of the heart pump as determined in act 352.

Process 350 may then proceed to act 360 where when it is determined that both the first flag and the second flag are set within a particular time window (e.g., 100 ms), process 350 may proceed to act 362, where it may be determined whether the magnitude of a shift in a pulsatility value (e.g., a normalized pulsatility value) of the motor current signal from a first time window before the analysis time window to a second time window after the analysis time window exceeds a third threshold value. The third threshold value may be determined in any suitable way. For example, the third threshold value may be empirically determined to minimize a number of false positives. In some embodiments, the third threshold value may be set to any suitable value. For example, the third threshold value may be set at a value of 0.1 mA, 0.2 mA, 0.3 mA, 0.4 mA, 0.5 mA, or some other suitable value.

The pulsatility value of the motor current signal may be determined in any suitable way. For instance, in some embodiments, the mean motor current (mean MC) value of the motor current signal within a time window may be calculated as:

$$mean MC = {\sum }_{i = 0}^{N} \frac{MC ( i )}{N}$$

where N represents the number of samples collected in the time window over which the mean MC value is calculated. In some embodiments, the time window over which mean MC is determined may be 100 ms, 150 ms, 200 ms, or some other suitable value.

The motor current signal pulsatility value may then be calculated as:

$$MC Signal Pulsatility = \frac{( max MC ) - ( min MC )}{mean MC} ,$$

where max MC is the maximum motor current value and min MC is the minimum motor current value within the time window in which mean MC is determined.

In some embodiments, the motor current signal pulsatility value may be normalized in any suitable way, and the normalized motor current signal pulsatility value may be used in the determination of act 362 in process 350.

If it is determined in act 362 that the motor current signal pulsatility value is greater than the third threshold value, process 350 may proceed to act 364, where it is determined whether a spike prominence value (e.g. of the spike in the motor current, observed at act 358) is greater than (or greater than or equal to) a fourth threshold value. In some embodiments, the spike prominence value may be determined as the average value of the motor current signal within a time window that starts at the peak of the motor current signal at spike 262 and ends a predetermined number of samples following the peak of the motor current signal spike 262. The predetermined number of samples may be any suitable value. For example, the predetermined number of samples may be 10, 11, 12, 13, etc. The predetermined number of samples may be empirically determined to minimize the number of false positives by selecting the predetermined number of samples to account for transient changes associated with the motor current signal.

An example of the spike prominence value of the motor current signal exceeding the fourth threshold value in act 364 may be visualized in FIG. 2B as spike 262 in the motor current signal 260 being followed by a sudden decrease in the motor current signal 260 following spike 262. The fourth threshold value may be determined in any suitable way. For example, the fourth threshold value may be determined to minimize a number of false positives by selecting a threshold value that differentiates between obstruction events and other heart pump related events (e.g., suction events) that may cause similar signal changes. In some embodiments, the fourth threshold value may be set to a value of 80 mA, 90 mA, 100 mA, 110 mA, or some other suitable value.

When it is determined in act 364 that the spike prominence value is greater than (or greater than or equal to) the fourth threshold value, process 350 may proceed to act 366, where it is determined whether a shift in a value (e.g., average value, average minimum value, average maximum value) of a motor current modulation signal (e.g. motor current modulation signal 280 shown in FIG. 2B) between a first time window before the analysis time window and the second time window after the analysis time window is greater than (or greater than or equal to) a fifth threshold value.

In some embodiments, the shift of the motor current modulation signal used in act 366 may be determined by comparing, a first motor current modulation value determined as an average motor current modulation signal value within a first time window prior to the analysis time window (e.g., time window 250) that does not include a time period (e.g., 30 seconds) immediately prior to the analysis time window, and a second motor current modulation value determined as an average motor current modulation signal value within a second time window following the analysis time window that does not include a time period (e.g., 30 seconds) immediately following the analysis time window according to:

$$Shift = ( \frac{AVG ( post outlier window ) - AVG ( pre - outlier window )}{AVG ( pre - outlier window )} ) \times 100$$

where, AVG(pre-outlier window) is the first motor current modulation value and AVG(post outlier window) is the second motor current modulation value.

If it is determined in act 366 that the shift of the motor current modulation signal is greater than the fifth threshold, an example of which is shown as shift 284 in FIG. 2B, a third flag may be set. In some embodiments, the fifth threshold value may be determined in any suitable way. For example, the fifth threshold value may be empirically determined to minimize a number of false positives. In some embodiments, the fifth threshold value may be set at a value of 5%, 10%, 15%, or some other suitable value.

Continuing with process 350, in act 368 a pressure signal (e.g., Pout) may be received from at least one pressure sensor associated with (e.g., arranged on) the heart pump. In some embodiments, the at least one pressure sensor may include an optical pressure sensor arranged near an outlet of the heart pump (e.g., to measure an aortic pressure). Process 350 may then proceed to act 370, where it is determined whether a shift of the value of the pressure signal before and after the analysis time window is greater than (or greater than or equal to) a sixth threshold value. In some embodiments, the shift in the value of the pressure signal used in act 370 may be determined by comparing a first pressure value (e.g., an average value, an average minimum value, an average maximum value) determined in a first time window before the analysis time window with a second pressure value (e.g., an average value, an average minimum value, an average maximum value) determined in a second time window after the analysis time window. An example of the shift in the pressure signal value exceeding the sixth threshold is illustrated as the shift 292 shown in FIG. 2B. In some embodiments, the sixth threshold value may be determined in any suitable way. For example, the sixth threshold value may be empirically determined to minimize a number of false positives. In some embodiments, the sixth threshold value may be set at a value of 5%, 10%, 15%, or some other suitable value. When it is determined in act 370 that the shift in the pressure signal value is greater than the sixth threshold, a fourth flag may be set.

Process 350 may then proceed to act 372 where it is determined whether the third and fourth flags are both set (e.g., within a particular time period). If it is determined in act 372 both of the third and fourth flags are set, it may be determined that an obstruction event has been detected, and process 350 may proceed to act 374, where an alarm may be output. An example of the third and fourth flags being set within a particular time period is shown by shift 284 and shift 292 exceeding the respective threshold for the same time period as shown in FIG. 2B. An indication of the obstruction event may be displayed in any suitable way. In some embodiments, an alarm may be displayed on a user interface associated with the heart pump (e.g., an alarm may be generated and displayed on the user interface) to indicate the detected obstruction event. Selecting a narrow time window (e.g., 120 ms, 100 ms, 80 ms, etc.) within which act 354, 358, 362, and 364 are performed may reduce false positives when detecting obstruction events using the techniques described herein.

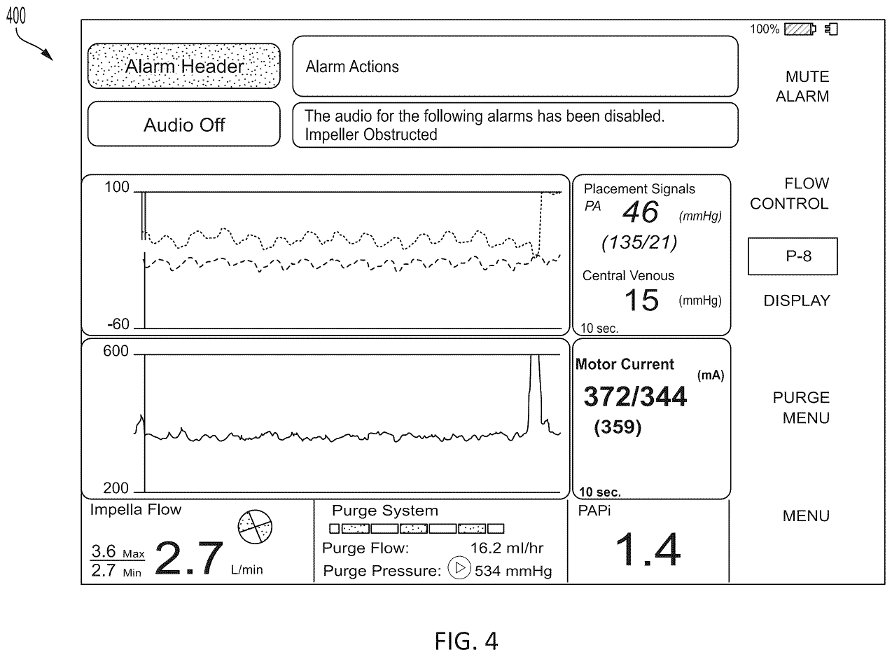

In some embodiments, values for at least some of the signals analyzed in process 300 (e.g. motor current signal, pump speed signal, pressure signal) and/or process 350 may be stored (e.g., in a log file) and retrieved from storage as desired for processing rather than being received directly from one or more sensors associated with the heart pump. As described herein, in some embodiments, after it is determined that an obstruction event has occurred, an indication of the obstruction event may be displayed on a user interface associated with the heart pump (e.g., on display 140 of the cardiac support system 120 shown in FIG. 1B). FIG. 4 shows an example of a user interface 400 on which such an indication may be displayed, in accordance with some embodiments. For example, user interface 400 may include one or more regions indicating various alarms that may be generated when certain operating conditions are met. In some embodiments, when an obstruction event is detected using one or more of the techniques described herein, an alarm for the obstruction event may be generated and displayed on a portion of a user interface, such as user interface 400.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of detecting an obstruction event associated with operation of a heart pump, the method comprising:
receiving a motor current signal associated with a motor of the heart pump;
receiving a pump speed signal associated with the heart pump;
detecting, using a computer processor, an obstruction event based, at least in part on:
a first value of the motor current signal being above a first threshold value at a first time; and
a second value associated with the pump speed signal being less than a second threshold value at a second time, wherein the first time and the second time are within a first time window having a length of 120 ms or less; and
displaying, when an obstruction event is detected, an indication of the obstruction event on a user interface associated with the heart pump.

2. The method of claim 1, further comprising:
receiving a pressure signal from at least one pressure sensor arranged on the heart pump; and
determining a first baseline shift of the pressure signal occurring within the first time window,
wherein detecting an obstruction event is further based, at least in part, on a value of the first baseline shift of the pressure signal being larger than a third threshold value.

3. The method of claim 2, wherein the heart pump is positioned across a valve in a right side of a patient's heart.

4. The method of claim 2, further comprising:
filtering the pressure signal to obtain a filtered pressure signal; and
determining the first baseline shift of the pressure signal based on the filtered pressure signal.

5. (canceled)

6. The method of claim 2, further comprising:
determining, based at least in part on the pressure signal, a type of obstruction event; and
displaying an indication of the type of obstruction event on the user interface associated with the heart pump.

7. The method of claim 2, further comprising:
determining a second baseline shift of the pressure signal occurring outside of the first time window; and
displaying on the user interface, an indication that the obstruction event has resolved when a value of the second baseline shift is larger than a fourth threshold value.

8. (canceled)

9. The method of claim 2, where determining a first baseline shift comprises comparing a current value of the pressure signal with a previous value of the pressure signal, wherein the previous value of the pressure signal occurred a predetermined number of samples prior to the current value.

10-12. (canceled)

13. The method of claim 1, further comprising:
receiving a pressure signal from a pressure sensor arranged on the heart pump;
computing a first value of the pressure signal within a second time window after an end of the first time window;
computing a second value of the pressure signal within a third time window before a start of the first time window; and
determining a first shift of the pressure signal based, at least in part, on the first value of the pressure signal and the second value of the pressure signal,
wherein detecting an obstruction event is further based, at least in part, on a value of the first shift of the pressure signal being greater than a third threshold value.

14. The method of claim 13, wherein:
the second time window begins an ignored time window after the end of the first time window, and
the third time window ends the ignored time window before the start of first time window.

15-16. (canceled)

17. The method of claim 13, wherein
computing the first value of the pressure signal within the second time window comprises determining the first value as an average minimum value of the pressure signal within the second time window, and
computing the second value of the pressure signal within the third time window comprises determining the second value as an average minimum value of the pressure signal within the third time window.

18. The method of claim 1, further comprising:
determining one or more derived signals based, at least in part, on the motor current signal and/or the pump speed signal,
wherein detecting an obstruction event is further based, at least in part, on a value of the one or more derived signals.

19. The method of claim 18, wherein the one or more derived signals includes a motor current amplitude modulation signal, and the method further comprises:
computing a first value of the motor current amplitude modulation signal within a second time window after an end of the first time window;
computing a second value of the motor current amplitude modulation signal within a third time window before a start of the first time window; and
determining a shift of the motor current amplitude modulation signal based, at least in part, on the first value and the second value,
wherein detecting an obstruction event is further based, at least in part, on a value of the shift of the motor current amplitude modulation signal.

20. The method of claim 19, wherein:
the second time window begins an ignored time window after the end of the first time window, and
the third time window ends the ignored time window before the start of the first time window.

21-22. (canceled)

23. The method of claim 18, wherein
the one or more derived signals includes a motor current signal pulsatility signal, and
detecting an obstruction event further based, at least in part, on a value of the motor current signal pulsatility signal being greater than a fifth threshold value.

24. The method of claim 23, further comprising:
generating a normalized motor current signal pulsatility signal based, at least in part, on the motor current signal pulsatility signal, wherein detecting an obstruction event further based, at least in part, on a value of the normalized motor current signal pulsatility signal.

25. The method of claim 1, wherein detecting an obstruction event is further based, at least in part, on a fourth value of the motor current signal being above a sixth threshold value.

26. The method of claim 25, further comprising:
computing the sixth threshold value based, at least in part, on a maximum value of the motor current signal during the first time window and a value of the motor current signal after the first time window.

27. The method of claim 25, further comprising:
computing the fourth value, wherein the fourth value is an average value of the motor current signal from a maximum value of the motor current signal during the first time window and a predetermined number of samples following the maximum value.

28. A heart pump system, comprising:
a heart pump including a rotor;
a motor configured to drive rotation of the rotor at one or more speeds; and
a controller configured to:
receive a motor current signal associated with the motor;
receive a pump speed signal associated with the heart pump;
detect an obstruction event based, at least in part on:
a first value of the motor current signal being above a first threshold value at a first time; and
a second value associated with the pump speed signal being less than a second threshold value at a second time, wherein the first time and the second time are within a time window having a length of 120 ms or less; and
display, when an obstruction event is detected, an indication of the obstruction event on a user interface associated with the heart pump.

29-50. (canceled)

51. A method of detecting an obstruction event associated with operation of a heart pump, the method comprising:
receiving a motor current signal associated with a motor of the heart pump;
receiving a pump speed signal associated with the heart pump;
receiving a pressure signal from a pressure sensor arranged on the heart pump;
determining one or more derived signals based, at least in part, on the motor current signal, the pump speed signal and/or the pressure signal, wherein the one or more derived signals includes a motor current amplitude modulation signal, and a motor current signal pulsatility signal;
computing a first value within a first time window of the pressure signal;
computing a second value within a second time window of the pressure signal;
computing a third value within the first time window of the motor current amplitude modulation signal;
computing a fourth value within the second time window of the motor current amplitude modulation signal;
determining a first shift of the pressure signal based, at least in part, on the first value of the pressure signal and the second value of the pressure signal;
determining a second shift of the motor current amplitude modulation signal based, at least in part, on the third value of the motor current amplitude modulation signal and the fourth value of the motor current amplitude modulation signal;
generating a normalized motor current signal pulsatility signal based, at least in part, on the motor current signal pulsatility signal;
detecting, using a computer processor, an obstruction event based, at least in part on:
a first value of the motor current signal being above a first threshold value at a first time,
a second value associated with the pump speed signal being less than a second threshold value at a second time, wherein the first time and the second time are within a third time window having a length of 120 ms or less,
a third value associated with the normalized motor current signal pulsatility signal being greater than a third threshold value,
a fourth value of the motor current signal being above a fourth threshold value, wherein the fourth value is a value of the motor current signal during the third time window,
a value of the first shift of the pressure signal being greater than a fifth threshold value, and
a value of the second shift of the motor current amplitude modulation signal being greater than a sixth threshold value; and
displaying, when an obstruction event is detected, an indication of the obstruction event on a user interface associated with the heart pump.
